(19) United States
(12) Patent Application Publication
(10) Pub. No.: US 2009/0202582 A1
(43) Pub. Date: Aug. 13, 2009

(54) Water-Disposable or Water-Soluble Polymer Film as a Vehicle for Dermatologic and Cosmetic Active Agents

(75) Inventors: Bernhard Irrgang, Menziken (CH); Reginald MacQuarrie, Toronto (CA)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: MIBELLE AG, Buchs (CH)

(21) Appl. No.: 12/367,042

(22) Filed: Feb. 6, 2009

(30) Foreign Application Priority Data
Feb. 7, 2008 (CH) 00164/08

(51) Int. Cl. A61K 36/02; A61K 36/00; A61K 31/728; A61K 47/38

(52) U.S. Cl. 424/195.17; 424/725; 514/54; 514/781

(57) ABSTRACT

Described are water-dispersible or water-soluble polymer films incorporating at least one cosmetic or dermatologic active agent and a method for the production thereof. The active agents involved are particularly anti-cellulite agents, moisturizers, anti-wrinkle agents and skin smoothing agents.

### BACKGROUND

The invention relates to a water-dispersible or water-soluble polymer film serving as a vehicle for dermatologic and cosmetic active agents. Fields of application include anti-cellulite treatment, skin moisturizing as well as skin firming and smoothing e.g. in reducing wrinkles.

Cosmetic products are available in a wealth of different forms. Highly effective cosmetic products are mostly emulsions which are applied to the skin and left there (“leave-on” products), it being here too, that the effect is most prominent. Products which are not left on the skin (“rinse off” products) are skin cosmetic formulations which have a lesser effect due to the limited contact time.

In general, “rinse off” products having a good effect fail to be available whilst although “leave-on” products often show a good effect they are hampered by other drawbacks.

Thus, WO 2006/009987 A2 describes a soluble film composition for application to the skin, it disclosing a system, a kit and a method with which an effective amount of an agent unstable in water can be applied to the skin.

Known from US 2003/0224053 are formulations and methods for liberating active agents to the skin. For this purpose a film is made available based on at least two polyvinyl alcohols of differing viscosity. Using this polyvinyl alcohol mixture does away with the need for a further polymer in creating a thin, transparent film.

US 2007/0154527A1 relates to self-supporting, fast-dissolving films and methods for their production, the films containing a topic agent for even distribution of the film. Because of how the film is formulated the active agents contained therein cannot enter into the blood circulation and thus have only a localized effect.

US 2004/0228892 discloses cosmetic and skin care products which are dispersed or enclosed in a polymer matrix. However, the polymer matrix needs to be dissolved or rendered molten before use so as to liberate the cosmetic or skin care product. Furthermore, use thereof in summer is not possible because of the low melting point.

Components of the emulsion remaining on the skin tend to result in irritations or because of their occlusivity block the pores of the skin, for example.

### SUMMARY AND DESCRIPTION

Accordingly, one object of the present invention is to create “rinse off” products having an enhanced active spectrum.

This object is achieved in accordance with the invention by a water-dispersible or water-soluble polymer film serving as a vehicle for dermatologic and cosmetic active agents.

Although water-soluble polymer films are known from the publication WO 2004/087857 they serve to apply surfactants, particularly soaps and detergents.

The product in accordance with the invention is a thin water-dispersible or water-soluble polymer film which in contact with water has intimate skin contact, the polymer film in general being completely dissolved with the further addition of water. Size and thickness of the polymer film are a function of the intended application.

Preference is also given to a polymer multilayer film featuring at least two water-dispersible or water-soluble polymer films each containing differing cosmetic or dermatologic active agents. After being applied to the skin this multilayer film, when brought into contact with water and because of the differing response of each polymer film to being dissolved, liberates the corresponding active agents one after the other.

The water-dispersible or water-soluble polymer film incorporating at least one cosmetic or dermatologic active agent comprises:

a plant-based polymer as the base component; and

1 to 40% by weight of a cosmetic or dermatologic active agent incorporated in the form of an emulsion, a gel or a solution in the base component.

### The Individual Components of the Polymer Film:

The base component, in other words the film polymer base, involves many different polymers suitable as the base for the production of rapid-dissolving films. Experience shows that natural polymers and carbon hydrate based hydrocolloids and/or proteins are best suitable. These include e.g. hydroxyl propylmethyl celluloses, sodium carboxy methyl celluloses, carboxy methyl celluloses, plant proteins such as soya proteins, gelatines (“high bloom to low bloom”) or milk proteins. Furthermore to be cited are alginates such as also hydroxylmethyl cellulose or hydroxylpropyl cellulose. But the film base may also consist of starches of differing mol weight or carrageenanes, various galactomannanes (“locust bean gum”), gums produced by microbial means such as xanthenes and gellane gum, as well as other various plant-based polymers such as guar or its derivatives, depending on the wanted orientation of the polymer film and depending on the wanted properties of how they dissolve in polar liquids such as water.

Other possible polymers include the group consisting of polyacrylic acids, polyethylene oxides and polyvinyl alcohols and their derivatives such as ether or ester.

The polymer film base may also be modified in its properties by the addition of polyolenes serving as a plasticizer, likewise by including emulsifiers and other auxiliary agents, with which the characteristic, properties and producibility of the polymer film can be tweaked.

Formulating Preparations with Active Agents:

The cosmetic or dermatologic active agent is worked into the base polymer in the form of an emulsion, a gel or a solution.

For preventive and active skin resurfacing, such as in the case of cellulite and for revitalising the skin a wealth of different active agents and combinations thereof are known. All known products of this kind can be incorporated in the aforementioned polymer films. These substances are intended e.g. to prevent or retard the development of cellulite. This is done by activating cell metabolism and also blood circulation. Likewise these substances serve to maintain or improve the firming and vital qualities of the skin.

To be cited in this respect are such active agents as e.g. caffeine, carnitine, algae extracts, e.g. spirulina platensia, plant and fruit extracts, e.g. from monk's pepper, butcher's broom, birch, white chestnut, tiger grass, ivy, horsetail to cite just a few. In addition to these vitamins such as folic acid, vitamin E. vitamins of the B series, vitamin C, ubiquinon (Q10) as well as minerals such as e.g. calcium, chrome, magnesium, potassium, manganese, molybdenum, selenium, iodine, zinc may be added singly or in combinations thereof. Also to be cited are such substances as are supportive in smoothing the skin, such as e.g. hyaluronic acid and vitamin A derivatives.

These active agents are ideally incorporated in the base formulation of the polymer film which may be either an emulsion, a gel or a solution.

In addition to this the polymer film contains:

(C) 1-30% by weight tensides and/or wetting agents

(D) 0.01-20% by weight modifiers

(E) 1-30% by weight moisturizers or oils and/or antimicrobic substances, fragrances, colorants

### Tensides and Wetting Agents:

A wealth of this class of substances may find application, including anionic, non-ionic, amphoteric tensides and combinations of substances of these groups, preference usually being given to the use of such popular tensides as sodium lauryl sulfates or sodium lauryl sulfates of ether. However, the wanted functionality of the product may necessitate including other or further tensides. Likewise possible is a further combination with emulsifiers and esters as usually employed in cosmetics, for example including secondary alkane sulfonates, cocoyl gluconates, lauroyl glutamates, methyl taurides, sodium cocyl isethionates, tributyl ether sulfates, C12-C16 alcohol ethoxylates, iso C10 alcohol ethoxylate, C11 alcohol ethoxylate or cocamido propyl betaine.

### Modifiers:

These include: colorants, agents as may be incorporated to impart a better skin feeling such as cationic polymers, silicones and natural oils as well as fragrances or stabilizers as per Attachment 6 of the EU Cosmetics Directive. Further auxiliary substances as may be incorporated for modifying the polymer film include antioxidants and vitamins, e.g. vitamins A, E or C as well as UV filters as per Attachment 2 of the EU Cosmetics Directive.

### Moisturizing Agents and Oils:

To be cited here are: emollient esters, silk protein derivatives and various phosphate esters, as well as aloe vera extracts, natural plant oils, soja oil derivatives. As also may be used are various fatty acids and their derivatives, cocoa butter, shea butter, coconut and palm kernel oils, canola oils and other natural oils.

These ingredients may be added directly to the slurry used in producing the polymer film.

### The Steps Typical for the Production Method:

Hot or cold water is added to the base formulation of the polymer film, depending on the nature of the polymer, to form a smooth, homogenous slurry.

In the next step, depending on the requirement and application, additional components such as tensides, oil, emulsifiers, stabilizers, moisturizers, plasticizers, colorants or fragrances are incorporated.

In the last step the wanted active agents are added to the preparation in continuous stirring.

The result is further stirred until a totally homogenous mass or liquid is obtained. Depending on the requirements profile an additional degasification step may be included to remove any trapped air.

The mass is then evenly spread over a heating band and dried down to the wanted liquid content. Typically a polymer film has a moisture content of 5 to 15%, ideally 3 to 10%.

Depending on the requirements of the production facilities and of the product the mass can be applied to the heating band in various ways, e.g. by means of mechanical spreaders, by extrusion or by rolling it on.

To assist finalizing the shape of the polymer film, gases such as air, nitrogen or other gases may be employed.

Depending on the requirements the polymer films may be further applied to other warmed up surfaces such as thin metal bands, bands of plastics (PP/PET), bands of coated paper (Mylar) for curing, it also being possible in this way to incorporate surface structures and colorations.

To prevent them sticking together the resulting polymer films may be powdered with such suitable substances as e.g. plant starch or talcum powder.

In conclusion the finished product may be cut and/or printed.

The polymer films as produced in this way can be further formed into tubes, sachets and other forms. For this purpose the polymer films may be welded by application of heat.

### The Polymer Films Feature the Following Physical Parameters:

Depending on the product specification the thickness of the polymer films can vary, normally in the range 15 to 600 microns.

The embrittlement or ultimate strength of the polymer films can be varied, it typically ranging from 2 to 50 psi (13790 to 344738 Pa).

Elasticity: This can be varied by employing plasticizers and adapting the base formulations as used.

### EXAMPLES

### Example 1

### Anti-Cellulite Film Type 1

| | | |
|---|---|---|
| water | 15 to 30% | by weight |
| carboxy methyl cellulose | 7 to 25% | by weight |
| alginates | 25 to 45% | by weight |
| preparation with active agents | 5 to 15% | by weight |
| tenside | 2.5 to 10% | by weight |
| emollients | 20 to 35% | by weight |
| fragrance | 1 to 8% | by weight |
| colorants | 0.01 to 0.20% | by weight |

### Example 2

### Anti-Cellulite Film Type 2

| | | |
|---|---|---|
| water | 10 to 25% | by weight |
| carboxy methyl cellulose | 15 to 35% | by weight |
| alginates | 5 to 20% | by weight |
| preparation with active agents | 10 to 30% | by weight |
| tenside | 7 to 20% | by weight |
| emollients | 20 to 35% | by weight |
| fragrance | 2 to 10% | by weight |
| colorants | 0.01 to 0.10% | by weight |

### Example 3

### Anti-Cellulite Film Type 3

| | | |
|---|---|---|
| water | 13 to 30% | by weight |
| carboxy methyl cellulose | 5 to 20% | by weight |
| alginates | 30 to 40% | by weight |
| preparation with active agents | 20 to 35% | by weight |
| tenside | 5 to 15% | by weight |
| emollients | 20 to 30% | by weight |
| fragrance | 1 to 7% | by weight |
| colorants | 0.02 to 0.10% | by weight |

### Example 4

### Anti-Cellulite Film Type 4

| | | |
|---|---|---|
| water | 15 to 30% | by weight |
| carboxy methyl cellulose | 10 to 25% | by weight |
| alginates | 10 to 25% | by weight |
| preparation with active agents | 25 to 35% | by weight |
| tenside | 5 to 20% | by weight |
| emollients | 5 to 15% | by weight |
| fragrance | 3 to 10% | by weight |
| colorants | 0.05 to 0.10% | by weight |

### Example 5

### Anti-Wrinkle Film Type 5

| | | |
|---|---|---|
| water | 10 to 25% | by weight |
| carboxy methyl cellulose | 25 to 30% | by weight |
| alginates | 5 to 15% | by weight |
| preparation with active agents | 10 to 15% | by weight |
| tenside | 2.5 to 25% | by weight |
| emollients | 15 to 35% | by weight |
| fragrance | 2 to 5% | by weight |
| colorants | 0.01 to 0.30% | by weight |

### Example 6

### Typical Preparation with Active Agents

| | | |
|---|---|---|
| water | 70 to 90% | by weight |
| ethanol | 0 to 10% | by weight |
| moisturizers | 1 to 15% | by weight |
| e.g. glycerine | | |
| or sorbitol | | |
| anti-cellulite agent 1 | 0.1 to 5% | by weight |
| e.g. caffeine | | |
| anti-cellulite agent 2 | 0.1 to 5% | by weight |
| e.g. Amara Shape | | |
| gelling agent | 0.1 to 3% | by weight |
| e.g. polyquaternium-37 | | |
| solutizer/emulsifier | 0.1 to 5% | by weight |
| e.g. polyglyceryl ester | | |
| or sorbitan ester | | |
| or fatty alcohol ethoxylates | | |
| aromatics | 0.01 to 5% | by weight |
| e.g. menthol | | |
| or peppermint oil | | |
| or orange oil | | |
| or other fruit oils | | |
| stabilizers | 0 to 0.8% | by weight |

### Example 7

### Active Agent Emulsion

| | | |
|---|---|---|
| water | 60 to 90% | by weight |
| ethanol | 0 to 10% | by weight |
| moisturizers | 1 to 15% | by weight |
| e.g. glycerine | | |
| or sorbitol | | |
| anti-wrinkle agent 1 | 0.001 to 3% | by weight |
| e.g. vitamin A palmitates | | |
| anti-wrinkle agent 2 | 0.0001 to 3% | by weight |
| e.g. Q10 | | |
| or sesame protein | | |
| or fruit extracts | | |
| plant oil | 0.1 to 15% | by weight |
| e.g. palm oil | | |
| or olive oil | | |
| or shea butter | | |
| or sesame oil | | |
| solutizer/emulsifier | 0.1 to 15% | by weight |
| e.g. polyglyceryl ester | | |
| or sorbitan ester | | |
| or benzyl/benzoate ester | | |
| fatty alcohol ethoxylates | | |
| or sugar derivatives | | |
| consistency agents | 0 to 5% | by weight |
| e.g. fatty alcohols | | |
| or glycerides | | |
| or phosphates | | |
| or starch derivatives | | |
| or cellulose derivatives | | |
| or guar derivatives | | |
| Aromatics | 0.01 to 5% | by weight |
| e.g. menthol | | |
| or peppermint oil | | |
| or orange oil | | |
| or other fruit oils | | |
| UV filter | 0 to 10% | by weight |
| stabilizers | 0 to 0.8% | by weight |

### Example 8

### Anti-Cellulite Film, Multilayer Type

base film

| | | |
|---|---|---|
| water | 15 to 30% | by weight |
| carboxy methyl cellulose | 10 to 25% | by weight |
| alginates | 10 to 25% | by weight |
| emollients | 5 to 15% | by weight |
| fragrance | 3 to 10% | by weight |
| colorants | 0.05 to 0.10% | by weight |

top layer

| | | |
|---|---|---|
| water | 15 to 30% | by weight |
| carboxy methyl cellulose | 10 to 25% | by weight |
| alginates | 10 to 25% | by weight |
| preparation with active agents | 1 to 15% | by weight |
| tenside | 5 to 20% | by weight |
| emollients | 5 to 15% | by weight |
| fragrance | 3 to 10% | by weight |
| colorants | 0.05 to 0.10% | by weight |

### Preferred Active Agents are:

| Product | Manufacturer |
|---|---|
| Amara Shape | Mibelle Biochemistry |
| Iso-Slim Complex | Mibelle Biochemistry |
| Sesaflash | Seppic |
| Perfelline | Rahn |
| Liftonin | Rahn |
| Cell-Activ series products | Rahn |
| Astraforce | Mibelle Biochemistry |
| Perfection Peptide P3 | Mibelle Biochemistry |

1. A water-dispersible or water-soluble polymer film incorporating at least one cosmetic or dermatologic active agent comprising
(A) a plant-based polymer as base component and
(B) 1 to 40% by weight of a cosmetic or dermatologic active agent incorporated in the form of an emulsion, a gel or a solution in the base component.

2. The polymer film as set forth in claim 1 wherein the base component is selected from the group consisting of carbon hydrate based hydrocolloids and/or proteins, plant proteins, starches, gelatines, alginates, carrageenanes, galactomannanes (locust bean gum), gums produced by microbial means, plant polymers.

3. The polymer film as set forth in claim 1 wherein the base component is selected from the group consisting of hydroxyl propylmethyl celluloses, sodium carboxy methyl celluloses, carboxy methyl celluloses, soya proteins, milk proteins, hydroxylmethyl celluloses, hydroxylpropyl celluloses, xanthenes, gellane, guar and their derivatives.

4. The polymer film as set forth in claim 1 wherein the dermatologic or cosmetic active agent is an anti-cellulite agent and/or a moisturizer and/or an anti-wrinkle agent and/or a skin smoothing agent.

5. The polymer film as set forth in claim 1 wherein the dermatologic or cosmetic active agent is an extract from plants and/or algae and/or vitamins and/or their derivatives and/or hyaluronic acid and/or its derivatives and/or minerals.

6. The polymer film as set forth in claim 1 wherein the polymer film comprises a moisture content of 5 to 15%, preferably 3 to 10%.

7. The polymer film as set forth in claim 1 characterized in that it rapidly dissolves in both cold and hot water.

8. The polymer film as set forth in claim 1 furthermore containing:
(C) 1.0-30% by weight tensides and/or wetting agents
(D) 0.01-20% by weight modifiers
(E) 1.0-30% by weight moisturizers or oils and/or antimicrobic substances, fragrances, colorants.

9. Use of the water-dispersible or water-soluble polymer film as set forth in claim 1 as a vehicle containing at least one dermatologic or cosmetic active agent for smoothing the skin, activating skin cell metabolism, activating blood circulation, for treatment of cellulite or for firming the skin.

10. Use of the water-dispersible or water-soluble polymer film as set forth in claim 1 in the form of a polymer multilayer film featuring at least two water-dispersible or water-soluble polymer films each containing differing cosmetic or dermatologic active agents.

11. A method for the production of a water-dispersible or water-soluble polymer film incorporating at least one cosmetic or dermatologic active agent, comprising the following steps in the method:
i. Adding hot or cold water to a base polymer, depending on the nature of the polymer, to form a smooth, homogenous slurry.
ii. Depending on the application, additional components selecting from the group consisting of tensides, oils, emulsifiers, stabilizers, moisturizers, plasticizers, colorants or fragrances are incorporated.
iii. Adding the dermatologic or cosmetic active components in the form of an emulsion, a gel or a solution to the resulting preparation.
iv. Stirring the resulting mixture until a totally homogenous mass or liquid is obtained.
v. Implementing a degasification step to remove any trapped air: and then
vi. Spreading the resulting mass evenly over a heating band and drying down to the wanted liquid content.

12. The method as set forth in claim 11, characterized in that the polymer film is dried to a moisture content of 5 to 15%, preferably 3 to 10%.

13. The method as set forth in any claim 11, characterized in that the mass is applied to the heating band by means of a mechanical spreader, by extrusion or by rolling it on.

14. The method as set forth in claim 11, characterized in that finalizing the shape of the polymer film is assisted by gases such as air, nitrogen or other inert gases.

15. The method as set forth in claim 11, characterized in that the polymer films are further applied to other warmed up surfaces such as thin metal bands, bands of PP/PET or paper bands coated with Mylar for curing.

16. The method as set forth in claim 15, characterized in that surface structures and tints are incorporated.

17. The method as set forth in claim 11, characterized in that to prevent them sticking together the resulting polymer films are powdered with a release agent such as plant starch or talcum powder.
